(19) United States
(12) Patent Application Publication
(10) Pub. No.: US 2008/0014614 A1
(43) Pub. Date: Jan. 17, 2008

(54) Labeling of rapamycin using rapamycin-specific methylases

(75) Inventors: Mark Edward Ruppen, Garnerville, NY (US); Pamela Fink Charbonneau, New City, NY (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/893,455

(22) Filed: Aug. 16, 2007

Related U.S. Application Data
(63) Continuation of application No. 11/113,686, filed on Apr. 25, 2005.
(60) Provisional application No. 60/565,714, filed on Apr. 27, 2004.

(51) Int. Cl. C12P 21/04; C12N 9/00

(52) U.S. Cl. 435/69.1; 435/183

(57) ABSTRACT

A method for rapamycin-specific labeling using rapI, rapM and/or rapQ enzymes is described. Also are methods for generating crude enzyme extracts useful in the method of the invention. Uses of the specifically labeled rapamycin as diagnostic tools are provided.

### CROSS-REFERENCE TO RELATED APPLICATIONS

This application is a continuation of U.S. patent application Ser. No. 11/113,686, filed Apr. 25, 2005, which claims the benefit under 35 USC 119(e) of prior U.S. Provisional Patent Application No. 60/565,714, filed Apr. 27, 2004.

### BACKGROUND OF THE INVENTION

Rapamycin is a macrocyclic triene antibiotic produced by Streptomyces hygroscopicus, which was found to have antifungal activity, particularly against Candida albicans, both in vitro and in vivo. [C. Vezina et al., J. Antibiot. 28, 721 (1975); S. N. Sehgal et al., J. Antibiot. 28, 727 (1975); H. A. Baker et al., J. Antibiot. 31, 539 (1978); U.S. Pat. No. 3,929,992; and US Patent No. 3,993,749]. The immunosuppressive effects of rapamycin have been described. FK-506, another macrocyclic molecule, has also been shown to be an immunosuppressive agent. These compounds have also been shown to be useful for a variety of other therapeutic indications. Rapamycin is commercially available under the Rapamune® name.

A rapamycin ester, rapamycin 42-ester with 3-hydroxy-2-(hydroxymethyl)-2-methylpropionic acid [described in U.S. Pat. No. 5,362,718], also known as CCI-779, has been shown to have antitumor activity against a variety of tumor cell lines, in in vivo animal tumor models, and in Phase I clinical trials. [Gibbons, J, Proc. Am. Assoc. Can. Res. 40: 301 (1999); Geoerger, B., Proc. Am. Assoc. Can. Res. 40: 603 (1999); Alexandre, J., Proc. Am. Assoc. Can. Res. 40: 613 (1999); and Alexandre, J, Clin. Cancer. Res. 5 (November Supp.): Abstr. 7 (1999)].

The labeling of rapamycin with labeling precursor compounds, including acetate, propionate or methionine [N. L. Paive and A. L. Demain, J. Natl Products, 54(1): 167-177 (January-February 1991)], or shikimic acid [P. A S. Lowden, et al, Angew. Chem. Int. Ed. 40(4):777-779 (2001)] by adding these compounds to fermentation cultures has been described. In these methods, as the bacteria synthesize rapamycin, some of the labeled material is incorporated into the newly produced rapamycin. The labeled rapamycin is purified from a mixture of other molecules, some of which might also carry the label. However, these methods provide inconsistent results in that not every rapamycin molecule isolated is labeled to the same extent, or in the same position.

What is desired are methods of specifically labeling rapamycin to produce a uniformly labeled molecule.

### SUMMARY OF THE INVENTION

The procedure described in the invention uses a specific methylase to label solely rapamycin in a uniform manner. The methylase, which is present in a crude cell extract, adds a labeled methyl group to purified desmethyl-rapamycin in vitro. In this system, rapamycin is the only molecule that is labeled. It may be tagged with isotopic labels, e.g., radioactivity. Isolation of the labeled material is quite simple using standard methods. Labeled rapamycin is readily identifiable based on its mass and/or radioactive label.

Other aspects and advantages of the invention will be readily apparent from the following detailed description of the invention.

### DETAILED DESCRIPTION OF THE INVENTION

The gene cluster responsible for the biosynthesis of rapamycin has been sequenced and analyzed [Schwecke et al., PNAS USA 92, 7839-43 (1995); Molnar et al., Gene 169, 1-7 (1996); Aparicio et al., Gene 169, 9-16 (1996)]. Following the synthesis and cyclization of the core polyketide, which is mediated by the protein products of rapA, rapB, rapC and rapP, further modifications are made to the molecule. Among these modifications are oxidations and methylations.

Three genes have been identified as S-adenosyl-L-methionine (SAM)-dependent methyltransferases, rapI, rapM and rapQ. RapI methylates the C-41 hydroxyl, and RapM and RapQ methylate the C-7 and C-32 hydroxyl groups [Chung et al., J. Antibiotics 54, 250-256 (2001)].

The method of the invention takes advantage of these rapamycin-specific methyl transferases (methylases) to efficiently label a desmethyl rapamycin in vitro.

Three enzymes, encoded by the genes, rapI, rapM and rapQ, are used in the method of the invention. These enzymes can be used individually, or mixtures thereof can be used in the process of the invention.

As defined herein, the term “a rapamycin” defines a class of immunosuppressive compounds which contain the following rapamycin nucleus:

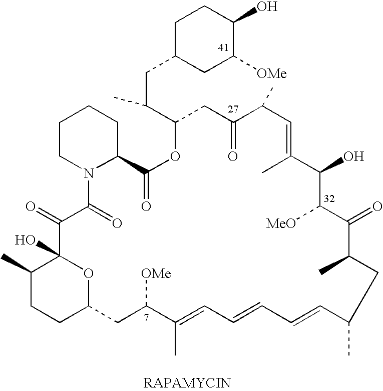

The term “desmethylrapamycin” refers to the class of immunosuppressive compounds which contain the basic rapamycin nucleus shown, but lacking one or more methyl groups. In one embodiment, the rapamycin nucleus is missing a methyl group from either positions 7, 32, or 41, or combinations thereof. The synthesis of other desmethylrapamycins may be genetically engineered so that methyl groups are missing from other positions in the rapamycin nucleus. Production of desmethylrapamycins have been described. See, e.g., 3-desmethylrapamycin [U.S. Pat. No. 6,358,969], and 1 7-desmethylrapamycin [U.S. Pat. No. 6,670,168].

The terms “desmethylrapamycin” and “—O-desmethylrapamycin” are used interchangeably throughout the literature and the present specification, unless otherwise specified.

The rapamycins used according to this invention include compounds which may be chemically or biologically modified as derivatives of the rapamycin nucleus, while still retaining immunosuppressive properties. Accordingly, the term “a rapamycin” includes esters, ethers, oximes, hydrazones, and hydroxylamines of rapamycin, as well as rapamycins in which functional groups on the nucleus have been modified, for example through reduction or oxidation. The term “a rapamycin” also includes pharmaceutically acceptable salts of rapamycins, which are capable of forming such salts, either by virtue of containing an acidic or basic moiety.

As used herein, pharmaceutically acceptable salts include, but are not limited to, hydrochloric, hydrobromic, hydroiodic, hydrofluoric, sulfuric, citric, maleic, acetic, lactic, nicotinic, succinic, oxalic, phosphoric, malonic, salicylic, phenylacetic, stearic, pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydroxymethyl)aminomethane. Additional pharmaceutically acceptable salts are known to those skilled in the art.

In one embodiment, the esters and ethers of rapamycin are of the hydroxyl groups at the 42- and/or 31 -positions of the rapamycin nucleus, esters and ethers of a hydroxyl group at the 27-position (following chemical reduction of the 27-ketone), and that the oximes, hydrazones, and hydroxylamines are of a ketone at the 42-position (following oxidation of the 42-hydroxyl group) and of 27-ketone of the rapamycin nucleus.

In another embodiment, 42- and/or 31-esters and ethers of rapamycin are described in the following patents: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 5,51,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers is described in the patents listed above.

In yet another embodiment, 27-esters and ethers of rapamycin are described in U.S. Pat. No. 5,256,790. The preparation of these esters and ethers is described in the patent listed above.

In still another embodiment, oximes, hydrazones, and hydroxylamines of rapamycin are described in U.S. Pat. Nos. 5,373,014, 5,378,836, 5,023,264, and 5,563,145. The preparation of these oximes, hydrazones, and hydroxylamines is described in the above-listed patents. The preparation of 42-oxorapamycin is described in U.S. Pat. No. 5,023,263.

In another embodiment, rapamycins include rapamycin [U.S. Pat. No. 3,929,992], rapamycin 42-ester with 3-hydroxy-2-(hydroxymethyl)-2-methylpropionic acid [U.S. Pat. No. 5,362,718], and 42-O-(2-hydroxy)ethyl rapamycin [U.S. Pat. No. 5,665,772]. The preparation and use of hydroxyesters of rapamycin, including CCI-779, is described in U.S. Pat. Nos. 5,362,718 and 6,277,983.

Although the examples provided herein illustrate methylation of 7-O-desmethyl-rapamycin [U.S. Pat. No. 6,399,626] and 32-O-desmethylrapamycin, these compounds are not a limitation of the invention.

1. The RapI, RapM, and RapQ Enzymes

In one embodiment, the rapamycin methylating enzymes defined herein are used in the form of crude enzyme extracts from Streptomyces hygroscopicus. In a further embodiment, crude enzyme extracts are prepared from S. hygroscopicus cells [available from the American Type Culture Collection, Manassas, Va., US, accession number ATCC29253, or from other sources]. In one embodiment, these cells are cultivated in shake flask fermentations using a method such as that described in Kim et al. (Kim, W-S. et al., 2000, Antimicrob. Agents Chemother. 44: 2908-2910). In another embodiment, for preparing cell free extracts, cells are collected by centrifugation, and about 1 gram of cell material is resuspended in about 20 mL of a suitable buffer. In yet another embodiment, the buffer is 50 mM 2-(N-morpholino)ethanesulfonic acid (MES) at a pH of about 6. In still another embodiment, the buffer is 50 mM potassium phosphate at a pH of 7.5. Cells are then disrupted and cell debris is removed by centrifugation. In one embodiment, supernatants are adjusted to 10% glycerol prior to freezing, e.g., at −70° C. In other embodiments, alternative methods for preparing crude enzyme extracts from the cell cultures will be readily apparent to one of skill in the art.

In yet another embodiment, these enzymes are further purified by classical protein isolation methods such as ammonium sulfate precipitation, column chromatography, etc.

In still another embodiment, the enzymes are synthesized by recombinant techniques, using classical in vitro transcription and translation methodologies.

The nucleic acid sequences of the rapI, rapM and rapQ enzyme genes are available from the PubMed NCBI on-line database, under accession No. X86780 for S. hygroscopicus. The nucleic acid sequences of the rapQ methylases gene are located at nt 90798-91433 of the CDS; protein ID# CAA60463.1 provides the amino acid sequence. The nucleic acid sequences of the rapM methylases gene are located on the complement of nt 92992-93945 of CDS; protein ID# CAA60466.1 provides the amino acid sequence. The nucleic acid sequences of the rapI methylases gene are located at nt 97622-98404 of the CDS, protein ID # CAA604701 provides the amino acid sequence. See, also, T. Schwecke, et al, Proc. Natl. Acad Sci. U.S.A. 92 (17), 7839-7843 (1995); I. Molnar, et al, Gene 169 (1), 1-7 (1996), and J. F. Aparicio, et al., Gene 169 (1), 9-16 (1996). The preceding nucleic acid and amino acid sequences are hereby incorporated by reference.

In another embodiment, the genes encoding the rapamycin methylation enzymes described herein are cloned into a suitable vector operably linked to regulatory control sequences that control expression thereof. As used herein, “operably linked” sequences include both expression control sequences that are contiguous with the gene of interest and expression control sequences that act in trans or at a distance to control the gene of interest. Expression control sequences include appropriate transcription initiation (promoter) and termination; sequences that enhance translation efficiency (e.g., Shine-Dalgarno site or ribosome binding site); and when desired, sequences that enhance secretion of the encoded product. A great number of expression control sequences, including promoters which are native, constitutive, and/or inducible, are known in the art and may be utilized.

In one embodiment, the regulatory control sequences include a regulatable or inducible promoter. Many such regulatable or inducible promoter systems have been described and are available from a variety of sources. Inducible promoters allow regulation of gene expression and can be regulated by exogenously supplied compounds, or environmental factors such as temperature. Inducible promoters and inducible systems are available from a variety of commercial sources, including, for example and without limitation, Invitrogen, Clontech and Ariad. Many other systems have been described and can be readily selected by one of skill in the art. For example, inducible promoters include the T7 polymerase promoter system [International Patent Publication No. WO 98/10088]. In one embodiment, the systems are selected for use in bacterial systems.

In another embodiment, as illustrated below, one or more of the genes encoding the enzyme are cloned into a commercial vector which expresses the enzyme(s) under an inducible promoter, i.e., pET24 inducible plasmid expression vector [Novagen]. However, one of skill in the art can readily select another vector and/or another suitable promoter for expression of the enzymes.

The vector may be any vector known in the art or described above, including naked DNA, a plasmid, phage, transposon, cosmids, episomes, viruses, etc. Introduction into the host cell of the vector may be achieved by any means known in the art or as described above, including transformation, transduction, and electroporation. Introduction of the molecules (as plasmids or viruses) into the host cell may also be accomplished using techniques known to the skilled artisan and as discussed throughout the specification. In one embodiment, standard transformation techniques are used, e.g., CaCl₂-mediated transformation or electroporation.

Once cloned into a suitable expression vector, the nucleic acid sequences encoding the enzyme are introduced into a suitable host cell for expression. In one embodiment, a suitable host cell is selected from prokaryotic (i.e., bacterial) cells. In the examples below, the host cells are Escherichia coli cells. However, one of skill in the art can readily select another appropriate host cell for expression of the selected enzymes.

In one embodiment, as illustrated below, crude enzyme extracts are prepared using recombinant techniques. [See, generally, Sambrook et al, Molecular Cloning: A Laboratory Manual, Cold Spring Harbor Press, Cold Spring Harbor, N.Y.] For example, a cell transduced with the rapamycin methylase genes is cultured under conditions that permit expression of the methylases(s). Where an inducible or regulatable protein, these conditions include supplying the inducing agent. Following culture, the cells are pelleted by centrifugation and resuspended in a suitable buffer, including a reducing agent, and phosphate buffer, adjusted to a neutral pH. In one embodiment, the buffer contains 50 to 100 mM potassium phosphate buffer, pH 7 to 7.5, containing 1 mM to 2 mM β-mercaptoethanol. In another embodiment, lysozyme is added at a final concentration of 100 μg/ml. In yet another embodiment, a suitable nuclease [e.g., BenzonaseTm nuclease] is added at 0.5-2.0 μL/mL cells. In still another embodiment, cell suspensions are incubated, e.g., for 15 minutes at 30° C. In yet another embodiment, a protease inhibitor (e.g., phenyl methyl sulfonyl fluoride (PMSF)) is added to the cells at a final concentration of 0.5-1.5 mM.

In a further embodiment, cells are fragmented by suitable means. In one embodiment, fragmentation is by mechanical means, e.g., by sonication on ice. Cell debris is removed and the resulting supernatants are adjusted to 5-15% glycerol (v/v) before freezing. The resulting crude enzyme extracts are now available for use in the rapamycin-specific methylation reaction of the invention.

In other embodiments, alternative methods for production and isolation of the enzymes will be readily apparent to those of skill in the art [Sambrook J et al. 2000. Molecular Cloning: A Laboratory Manual (Third Edition), Cold Spring Harbor Press, Cold Spring Harbor, N.Y.].

The methods for production, purification, and isolation are not limitations of the present invention.

II. The Methylation Reaction

Using a rapamycin methylase as described herein, either as a crude extract or another suitable form, the methylation reaction is performed as follows. Approximately 45 to 65% v/v crude methylase extract is added to a reaction containing about 8-130 μM desmethyl rapamycin solution, about 0.2-0.4 mM methylating reagent, about 4-10 mM magnesium (Mg, e.g., MgSO₄), and a suitable buffer at about 50-100 mM concentration, adjusted to a pH of 6.5 to 7.5. In another embodiment, a more purified form of the methylases is utilized in lower volume, e.g., about 10 to about 45% v/v methylase.

In one embodiment, the methyl donor is S-adenosyl-L-methionine (SAM). When selected for use in the invention, SAM is generally present at a final concentration of about 0.2-0.4 mM.

In another embodiment, a rapamycin solution is about 0.5 mg/mL to about 5 mg/mL, about 1 mg/mL to 3 mg/mL, or about 1 mg/mL rapamycin in a suitable solvent. Suitable solvents for the selected rapamycin include methanol, ethanol and dimethylformamide, tetrahydrofuran, or mixtures thereof.

Suitable buffers can be readily selected from among physiologically compatible buffers, including, e.g., phosphate buffered saline, a 2-(N-Morpholino)-ethanesulfonic acid (MES) buffer, Tris-(hydroxymethyl)aminomethane (Tris) buffer, or potassium phosphate buffer.

Following mixture of these components, the reaction is allowed to proceed. The 30 reaction temperature can vary from 20° C. to about 37° C. for about 0.5 to 3 hours, or about 1 to 2 hours. In one embodiment, the reaction mixture is incubated at about 34° C. for approximately 1 hour.

At the end of incubation, 1 to 2 volumes of a quenching reaction is added to terminate the reaction, e.g., ethanol, methanol or ethyl acetate.

Precipitated material is removed by conventional methods. In one embodiment, the precipitated material is removed by centrifugation. In a further embodiment, centrifugation is conducted at 14,000 rpm for 10 minutes. However, other removal methods and/or centrifugation conditions are known in the art.

Purification can be accomplished by any suitable method known to those of skill in the art. Suitable methods include recrystallization, silica gel column chromatography, thin layer chromatography (TLC) and high performance liquid chromatography (HPLC). In one embodiment, HPLC analysis is performed using a C18 column (3.9×150 mm) at 45° C. with a mobile phase comprised of 60% dioxane, 0.05% acetic acid and 0.03% triethylamine. In another embodiment, HPLC analysis is performed with a C18 column (4.6×250 mm) using a mobile phase gradient of 40%A:60%B going to 15%A:85%B over 75 minutes, where solvent A is 10 mM ammonium acetate in water and solvent B is methanol.

III. Composition and Uses

Labeled rapamycin is needed to study and/or monitor the metabolic fate of rapamycin in the body. In one embodiment, labeled rapamycin is used to identify cells/structures that have bound to rapamycin. Rapamycin may be uniformly tagged with either density or radioactive labels. Rapamycin labeled in the manner described will have the conformation and properties of unlabeled, native rapamycin, but is easily detectable because of the consistently incorporated density or radioactive label.

In one embodiment, the invention provides kits for specific labeling of rapamycin, comprising one or more of the enzymes described herein. The kits may further contain additional components, such as, e.g., a positive control (e.g., a methylated rapamycin), a negative control, reagents (e.g., buffer, lysozyme, nuclease), vials, tubes, and instructions for performing the method of the invention.

In certain circumstances, it is desirable to deliver the labeled rapamycin produced according to the present invention in a composition comprising a physiologically compatible carrier. These compositions are advantageous in that the labeled rapamycin compounds produced according to the invention can be readily tracked (i.e., monitored) using techniques known to those of skill in the art, e.g., mass spectrometry or scintillation counting, among others.

The following examples are illustrative of the methods of the invention for rapamycin specific methylation. It will be readily understood from a reading of the detailed description of the invention these examples do not limit the invention to the reaction conditions and reagents illustrated.

### EXAMPLES

A. Amplification of Methylase Genes

The genes were amplified from genomic S. hygroscopicus ATCC29253 DNA with oligonucleotide primers designed using the published rapamycin gene cluster sequence (Schwecke, T. et al., 1995, Proc. Natl. Acad. Sci. USA 92: 7839-7843). The RapI, RapM and RapQ proteins were then expressed in E. coli strain BL21(DE3) cells using the Novagen pET24 inducible plasmid expression vector. In this vector, cloned genes are expressed from a T7 promoter by T7 RNA polymerase, and expression is activated by IPTG addition.

B. Preparing Enzyme Extracts

To establish optimal conditions for an in vitro methylation reaction, crude enzyme extracts were prepared from S. hygroscopicus [ATCC29253] cells cultivated in shake flask fermentations using a method like that described in Kim et al. (Kim, W-S. et al., 2000, Antimicrob. Agents Chemother. 44: 2908-2910). Cells were collected by centrifugation, washed in 0.2 M MES buffer, pH 6.0, and cell pellets were frozen prior to extraction. 20 Approximately 8 g to 10 g of thawed cell material was resuspended in 20 mL of 50 mM MES buffer, pH 6.0. For crude extracts of the cloned methylase proteins, 25 mL cultures of induced cells were collected by centrifugation and the pellets frozen. The pellets were resuspended in 10 mL 50 mM potassium phosphate buffer, pH 7.5, containing 1 mM β-mercaptoethanol. Thereafter, lysozyme was added to a final concentration of 100 μg/ml and Benzonase™ nuclease was added (1 μL/mL cells). Cells were sonicated for 1 to 2 min on ice and cell debris was removed by centrifugation at ˜30,000×g, 4° C. for 15 min. Supernatants were adjusted to ˜10% glycerol prior to freezing at −70° C.

C. Methylation of 7-Desmethyl-rapamycin

Approximately 65 μL of crude methylase extract was added to a reaction containing 3 μL of 1 mg/mL 7-desmethyl- rapamycin (7-dmr) solution, 5 μL of 4 mM SAM, 4 μL of 0.1 M MgSO₄, and 23 μL of a 0.5 M phosphate buffer, adjusted to pH 7.5. Methylation reactions using the recombinant cell extracts were carried out as described above, except that 50 μL of extract and 38 μL of buffer were used. HPLC chromatograms from reactions containing the RapM methylase extract and two desmethyl-rapamycin substrates, 7-O-desmethyl-rapamycin (7-dmr and 32-O-desmethyl-rapamycin show that the RapM methylase generated rapamycin (rapa) only when 7-dmr was the substrate. The enzyme did not modify 32-dmr, indicating that the cloned enzyme retained its substrate specificity in vitro. In addition, samples with no SAM added showed no conversion of 7-dmr to rapamycin.

D. Labeling Rapamycin with Methyl-tritiated SAM

For labeling of rapamycin, the same type of in vitro reaction was used. For example, reaction mixtures for RapM methylation contained the following: 10 μL 0.5 M KPO₄ buffer, pH 7.5, 4 μL of 0.1 μM MgSO₄, 33 μL of 100 μM S-adenosyl-L-methionine-(methyl-³H), 3 μL of 1 mg/mL 7-desmethyl-rapamycin (in ethanol), and 50 μL crude extract. The following scheme shows an example of the labeled rapamycin molecule that would be generated by the action of the RapM methylase on the 7-dmr substrate.

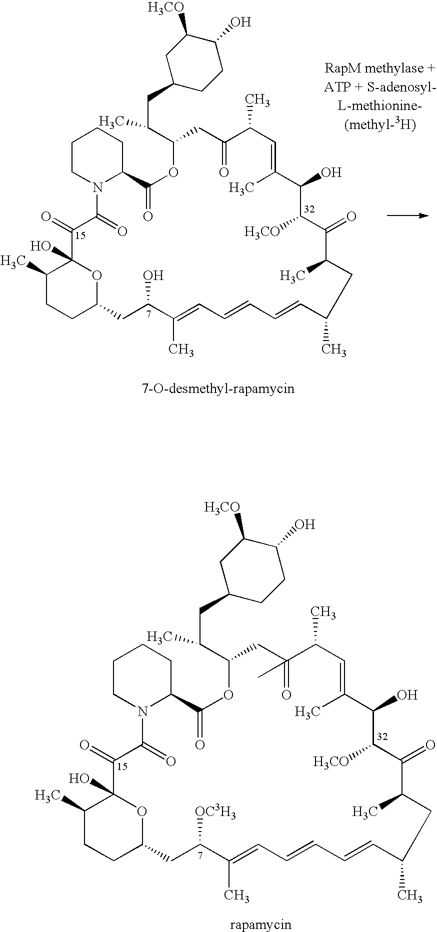

The tritiated material was indistinguishable from the rapamycin standard by HPLC analysis. Mass spectral data indicated that the labeled material was consistent with tritiated rapamycin.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

Patents, patent applications, publications, procedures, and the like are listed throughout this application, the disclosures of which are incorporated herein by reference in their entireties. To the extent that a conflict may exist between the specification and another document, the language of the disclosure made herein controls.

1. A method for specifically labeling a rapamycin comprising the step of:
reacting a desmethylrapamycin with a rapamycin-specific methylase in the presence of a methylating reagent.

2. The method according to claim 1, wherein the methylating reagent is S-adenosyl-L-methionine.

3. The method according to claim 1, wherein the methylase is selected from the group consisting of rapI methylase, rapM methylase, and rapQ methylase.

4. The method according to claim 1, wherein the methylase is in the form of a crude enzyme extract.

5. The method according to claim 4, wherein the crude enzyme extract is prepared by the steps comprising:
(a) expressing the rapamycin-specific enzyme from a cell culture transduced with a nucleic acid sequence encoding an enzyme operably linked to regulatory control sequences, said enzyme selected from the group consisting of rapI methylase, rapM methylase and rapQ methylase;
(b) concentrating the cells and resuspending them in a buffer;
(c) incubating the mixture with lysozyme and nuclease;
(d) fragmenting the cells; and
(e) centrifuging and collecting the supernatant.

6. The method according to claim 5, wherein the incubation in step (c) further comprises a protease inhibitor.

7. The method according to claim 1, wherein the reaction mixture is incubated at about 34° C. for about 1 hour.

8. The method according to claim 1, wherein methanol is added to the reaction at the end of incubation.

9. The method of claim 1, wherein precipitated material is removed by centrifugation prior to HPLC analysis.

10. The method of claim 9, wherein HPLC analysis is performed in a C18 column at 45° C. with a mobile phase comprising dioxane, acetic acid and triethylamine.

11. A specifically labeled rapamycin produced according to the method of claim 1.

12. A composition comprising a specifically labeled rapamycin produced according to the method of claim 1 and a physiologically compatible carrier.

13. A kit for producing a specifically labeled rapamycin comprising a methylated rapamycin produced according to the method of claim 1, and one or more components selected from the group consisting of a negative control, a methylation reagent, a vial, a tube, and instructions.

14. A kit comprising the labeled rapamycin according to claim 11.
